(19) United States
(12) Patent Application Publication
(10) Pub. No.: US 2012/0178821 A1
(43) Pub. Date: Jul. 12, 2012

(54) POLYMORPHIC FORM OF TOREMIFENE CITRATE AND PROCESS FOR ITS PREPARATION

(75) Inventors: Pranab Chatterjee, Nadia (IN); Asok Nath, Gurgaon (IN); Mohan Prasad, Gurgaon (IN)

(73) Assignee: RANBAXY LABORATORIES LIMITED, New Delhi, Delhi (IN)

(21) Appl. No.: 13/388,134

(22) PCT Filed: Jul. 30, 2010
(86) PCT No.: PCT/IB10/53488

§ 371 Date: Mar. 30, 2012

(30) Foreign Application Priority Data
Jul. 31, 2009 (IN) 1593/DEL/2009

(51) Int. Cl. A61K 31/138; C07C 59/265

(52) U.S. Cl. 514/648; 562/584

(57) ABSTRACT

The present invention provides a polymorphic form of toremifene citrate and processes for its preparation. It also relates to an improved process for the preparation of the Z isomer of the toremifene base, free of E isomer, and its pharmaceutically acceptable salts.

### FIELD OF THE INVENTION

The present invention provides a polymorphic form of toremifene citrate and processes for its preparation. It also relates to an improved process for the preparation of the Z isomer of the toremifene base, free of E isomer, and its pharmaceutically acceptable salts.

### BACKGROUND OF THE INVENTION

Toremifene, the Z isomer of 2-(p-[4-chloro-1,2-diphenyl-1-butenyl]-phenoxy)-N,N-dimethylethylamine, is a triphenylethylene derivative of formula given below:

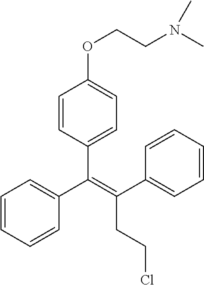

Toremifene has antiestrogenic activity and is useful in the treatment of hormone dependent breast cancer. A method of preparing toremifene, its analogs and salts has been described in U.S. Pat. No. 4,696,949 (herein after referred to as “'949 Patent”).

The '949 Patent describes a process for preparing toremifene citrate, that includes dissolution of free toremifene base in warm acetone, addition of citric acid solution in acetone to the reaction mixture, followed by cooling and isolation. It further discloses the citrate salt of the toremifene being characterized by its melting point (160-162° C.). No additional characterization data is disclosed.

The '949 Patent also discloses the preparation of toremifene base or its analogues in pure Z form involving resolution of the Z:E mixture of 1,2-diphenyl-1-[4-[2-(N,N-dimethylamino)ethoxy]-phenyl]-1-buten-4-ol or its corresponding analogues and their reaction with thionyl chloride. The process includes either:
(a) isolation of Z and E isomers of 1,2-diphenyl-1[4-[2-(N,N-dimethylamino)ethoxy]-phenyl]-1-buten-4-ol or analogue through crystallization from solvents such as hexane-ethanol (95:5), toluene-petrol ether (1:1) and toluene; or
(b) isolation of Z and E isomers through salt formation, particularly the hydrochloride salt in ethanol with concentrated hydrochloric acid or gas followed by double recrystallization in alcohol and finally in a ketone, specifically acetone.

WO 2004/101492 describes the preparation of toremifene base or a pharmaceutically acceptable salt, free of E isomer, involving a two step crystallization process. The first solvent is methanol and the second solvent is selected from acetone, methyl ethyl ketone or ethyl acetate, which can be converted to pharmaceutically acceptable salt, if desired.

Until now, the prior art does not provide any other reference related to the crystalline form of toremifene citrate characterized by its XRD, IR and DSC. The only reference is in the basic patent ('949 Patent), which describes a crystalline toremifene citrate characterized by only its melting point. Also the methods reported in literature for the preparation of Z-isomer of the toremifene base and its pharmaceutically acceptable salts from the Z:E isomer mixture of the toremifene analogs either involves double or several recrystallizations from one or more solvents or involves a salt formation followed by recrystalizations in multiple solvents.

Accordingly there is a need for new crystalline forms of toremifene citrate having better dissolution to meet the requirements of enhanced bioavailability much needed for formulation.

The present inventors have now found a polymorphic form of toremifene citrate. Also the present inventors have developed a process for the preparation of toremifene base and its pharmaceutically acceptable salts which is simple, cost effective and industrially advantageous and overcomes the drawbacks of prior art processes.

### SUMMARY OF THE INVENTION

In one general aspect, the present invention provides for polymorphic Form II of toremifene citrate.

In another general aspect, the present invention provides for polymorphic Form II of toremifene citrate that has substantially the equivalent XRPD pattern as depicted in FIG. 1.

In yet another general aspect, the present invention provides for polymorphic Form II of toremifene citrate which includes X-ray diffraction peaks expressed in degrees two-theta at 18.15, 18.88, 20.02, 21.39±0.2 in XRPD.

Embodiments of the present invention may include one or more of the following features. For example, the polymorphic Form II of toremifene citrate of may further include X-ray diffraction peaks expressed in degrees two-theta at 5.67, 8.46, 9.51, 10.45, 11.40, 12.48, 13.48, 14.27, 16.14, 17.09, 25.10, 26.37, 33.96±0.2 in XRPD.

In another general aspect, the present invention provides for polymorphic Form II of toremifene citrate having purity greater than 99.9% as measured by HPLC area percentage as shown in FIG. 4.

In yet another general aspect, the present invention provides for polymorphic Form II of toremifene citrate in Z isomer, which is free of E isomer.

In another general aspect, the present invention provides for a pharmaceutically acceptable composition that includes polymorphic Form II of toremifene citrate and one or more pharmaceutically acceptable carriers.

In another general aspect, the present invention provides for a method of treating or preventing hormone dependent tumors, wherein such method includes administering to a mammal a pharmaceutical composition comprising a therapeutically effective amount of polymorphic Form II of toremifene citrate.

In another general aspect there is provided a process for the preparation of polymorphic Form II of toremifene citrate. The process includes:
(a) treating toremifene base with a ketone at an ambient temperature to obtain a solution;
(b) adding citric acid to the solution of step (a); and
(c) isolating polymorphic Form II of the toremifene citrate.

Embodiments of the process may include one or more of the following features. For example, the ketone may include one or more of aliphatic or alicyclic ketones. The aliphatic ketones may include acetone, 2-pentanone, 3-pentanone, methylisobutyl ketone or methyl ethyl ketone. The alicyclic ketones may include cyclopentanone or cyclohexanone.

In another general aspect, the present invention provides for a process for the preparation of the Z-isomer of toremifene base or its pharmaceutically acceptable salts. The process includes the steps of:
(a) converting 1,2-diphenyl-1[4[-2-N,N-dimethylamino)ethoxy]-phenyl-1-butane1,4-diol to toremifene base without isolation of any intermediates;
(b) purification of crude toremifene base in ketonic solvent; and
(c) isolating the Z-isomer of toremifene base, which is optionally converted to pharmaceutically acceptable salts.
   Embodiments of the process may include one or more of the following features. For example, the ketone may include aliphatic or alicyclic ketones. The aliphatic ketone may include acetone, 2-pentanone, 3-pentanone, methylisobutyl ketone or methyl ethyl ketone. The alicyclic ketone may include cyclopentanone or cyclohexanone.

The 1,2-diphenyl-1[4-[2-N,N-dimethylamino)ethoxy]-phenyl-1-butane1,4-diol is converted to toremifene base without isolation of 4-hydroxy-1,2-diphenyl-1[4-[2-N,N-dimethylamino)ethoxy]-phenyl-1-butene intermediate.

The 1,2-diphenyl-1[4-[2-N,N-dimethylamino)ethoxy]-phenyl-1-butane1,4-diol is converted to toremifene base without isolation of 4-acetoxy-1,2-diphenyl-1[4-[2-N,N-dimethylamino)ethoxy]-phenyl-1-butene intermediate.

The 1,2-diphenyl-1[4-[2-N,N-dimethylamino)ethoxy]-phenyl-1-butane-1,4-diol is converted to toremifene base without isolation of 4-acetoxy-1,2-diphenyl-1[4-[2-N,N-dimethylamino)ethoxy]-phenyl-1-butanol intermediate.

In another general aspect, the present invention provides for toremifene base of purity greater than 99.9% as measured by HPLC area percentage.

In a final general aspect, the present invention provides for Z isomer of toremifene base which is free of E isomer.

### BRIEF DESCRIPTION OF THE DRAWINGS

FIG. 1: X-ray diffraction pattern for Form II of toremifene citrate.

FIG. 2: IR spectrum for Form II of toremifene citrate.

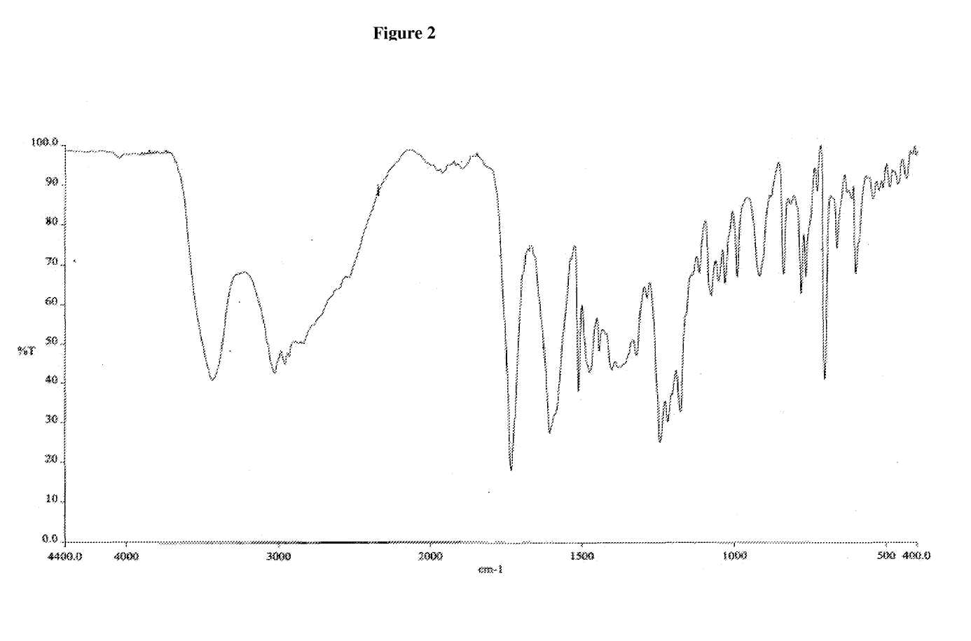

FIG. 3: DSC pattern for Form II of toremifene citrate.

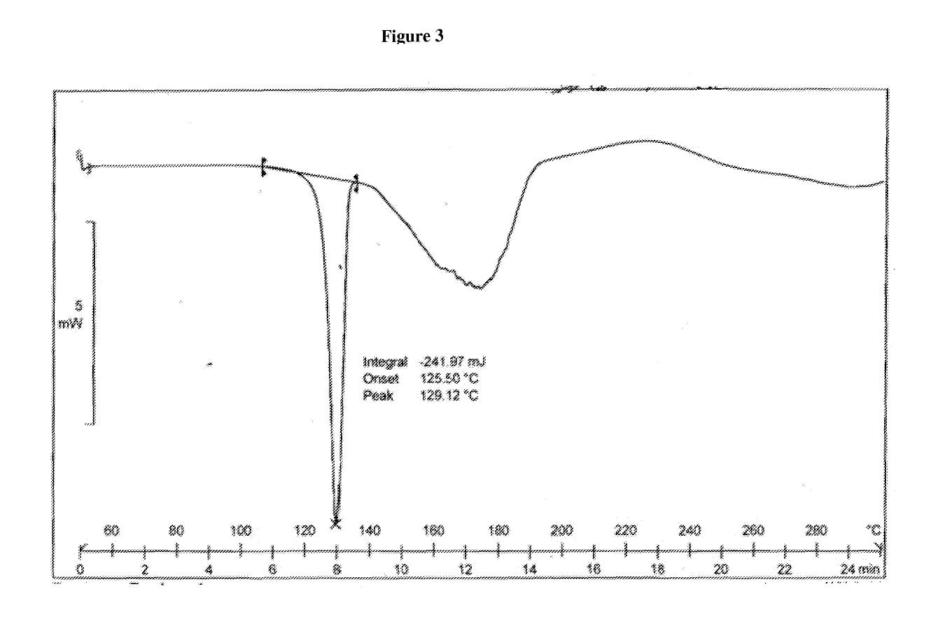

FIG. 4: HPLC chromatogram for Form II of toremifene citrate.

FIG. 5: X-ray diffraction pattern for toremifene citrate as per the '949 Patent.

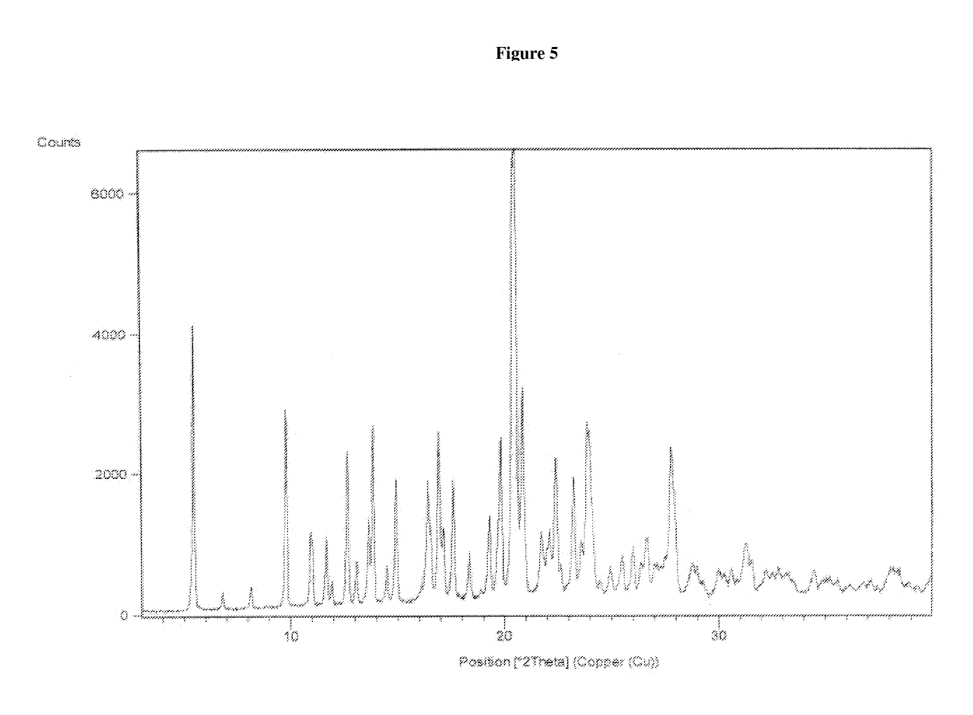

FIG. 6: IR pattern for toremifene citrate as per the '949 Patent.

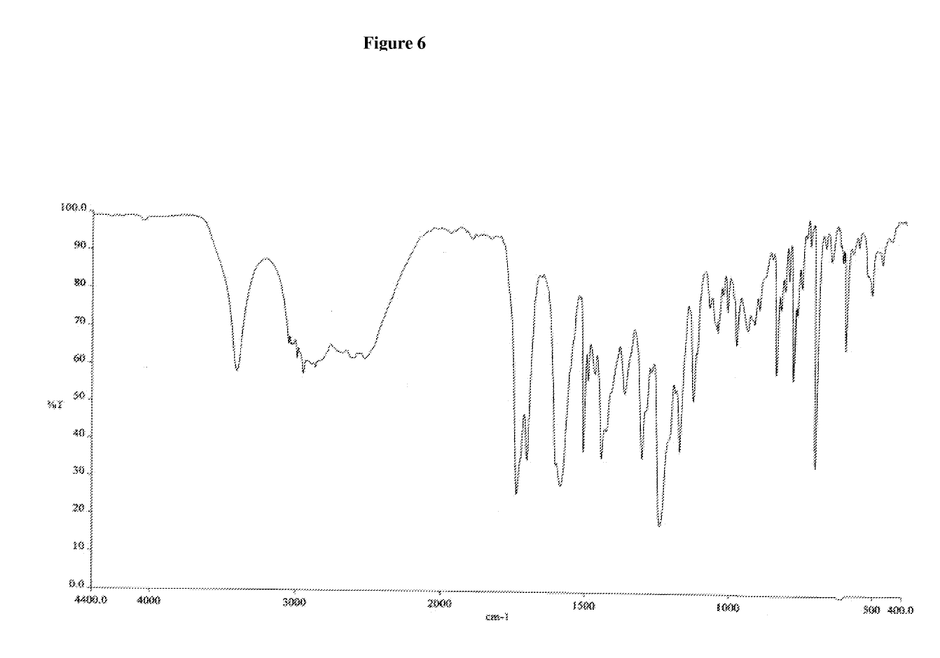

FIG. 7: DSC pattern for toremifene citrate as per the '949 Patent.

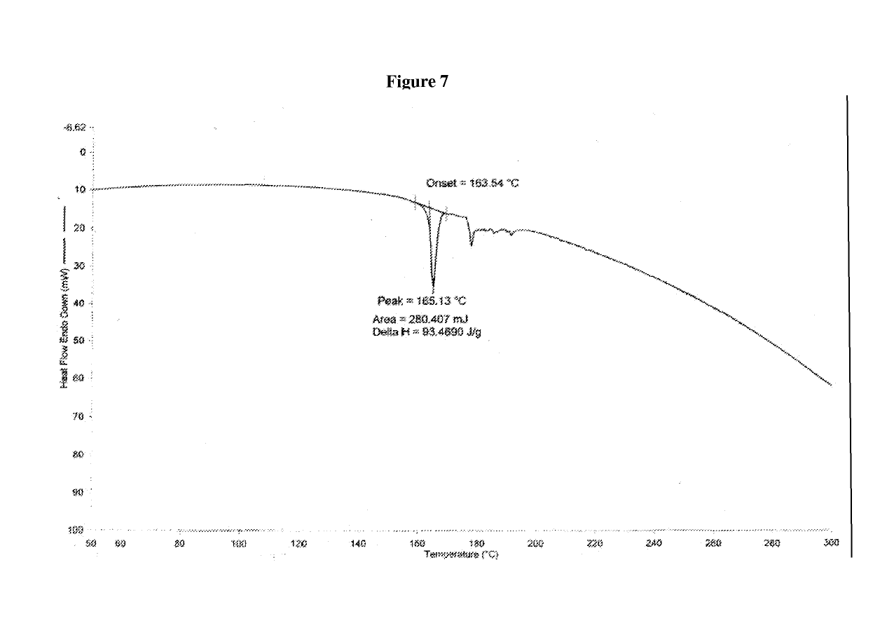

### DETAILED DESCRIPTION OF THE INVENTION

The XRPD of the polymorphic form of toremifene citrate prepared as per the '949 Patent shows characteristics d-spacing [Å] values at 2.344, 2.361, 2.421, 2.483, 2.524, 2.602, 2.732, 2.779, 2.834, 2.859, 2.923, 2.981, 3.105, 3.194, 3.215, 3.307, 3.353, 3.385, 3.427, 3.495, 3.570, 3.639, 3.732, 3.774, 3.830, 3.971, 4.025, 4.097, 4.263, 4.324, 4.356, 4.474, 4.599, 4.835, 5.036, 5.166, 5.238, 5.396, 5.924, 6.104, 6.382, 6.489, 6.762, 6.983, 7.421, 7.575, 8.090, 9.010, 10.860, 12.967, and 16.137. This polymorphic form is designated herein as Form I, which is characterized by its XRD, DSC and IR.

The corresponding X-ray diffraction of Form I expressed in degrees two-theta are 5.47, 6.81, 8.14, 9.81, 10.93, 11.68, 11.92, 12.67, 13.09, 13.64, 13.87, 14.50, 14.95, 16.42, 16.92, 17.16, 17.60, 18.34, 19.29, 19.84, 20.38, 20.53, 20.83, 21.68, 22.08, 22.38, 23.22, 23.56, 23.84, 24.46, 24.94, 25.48, 25.99, 26.32, 26.57, 26.95, 27.74, 27.93, 28.74, 29.96, 30.57, 31.27, 31.57, 32.21, 32.77, 34.45, 35.56, 36.16, 37.13, 38.07, and 38.45.

The present invention provides a polymorphic Form II of the toremifene citrate characterized by its XRD, IR and DSC.

The XRPD of the Form II of toremifene citrate shows characteristics d-spacing [Å] values at 2.639, 3.378, 3.546, 4.153, 4.435, 4.699, 4.885, 5.188, 5.490, 6.204, 6.564, 7.091, 7.758, 8.464, 9.293, 10.444, and 15.577.

The corresponding X-ray diffraction peaks of Form II expressed in degrees two-theta for Form II are 5.67, 8.46, 9.51, 10.45, 11.40, 12.48, 13.48, 14.27, 16.14, 17.09, 18.15, 18.88, 20.02, 21.39, 25.10, 26.37, and 33.96±0.2. The DSC of the Form II of the toremifene citrate shows characteristic endotherm in the range of 125-29° C.

Form II of the present invention is Z isomer, which is free from E isomer, and has purity greater than about 99.9% when measured by HPLC area percentage.

Another aspect of the present invention provides a process for the preparation of polymorphic Form II of the toremifene citrate, wherein the process includes:
(a) treating toremifene base with a ketone at an ambient temperature to obtain a solution;
(b) adding citric acid to the solution; and
(c) isolating polymorphic Form II of toremifene citrate.

The citric acid may be added to the solution of toremifene base in ketone at the ambient temperature or vice versa. Ambient temperature here refers to room temperature.

Examples of ketone may include aliphatic or alicyclic ketones. Examples of aliphatic ketones may include acetone, 2-pentanone, 3-pentanone, methylisobutyl ketone and methyl ethyl ketone. Examples of alicyclic ketones may include cyclopentanone and cyclohexanone.

The citric acid can be added to the above reaction solution of toremifene base in ketone in solid form or in solution in the same ketonic solvent. The citric acid may be added to the above solution slowly over a time period from about 30 minutes to about 60 minutes. After the addition of citric acid the solution may be stirred at the same temperature until a solid precipitates out.

The solid obtained after stiffing can be isolated from the above reaction mixture through simple filtration, filtration under vacuum, evaporation, or distillation. Filtration is preferred for the isolation of the solid material. The solid is then dried by comprising air drying, vacuum drying or drying under nitrogen atmosphere. Preferably, vacuum drying is used.

The polymorphic Form II of the toremifene citrate so obtained has been characterized by the XRD, IR and DSC as mentioned above.

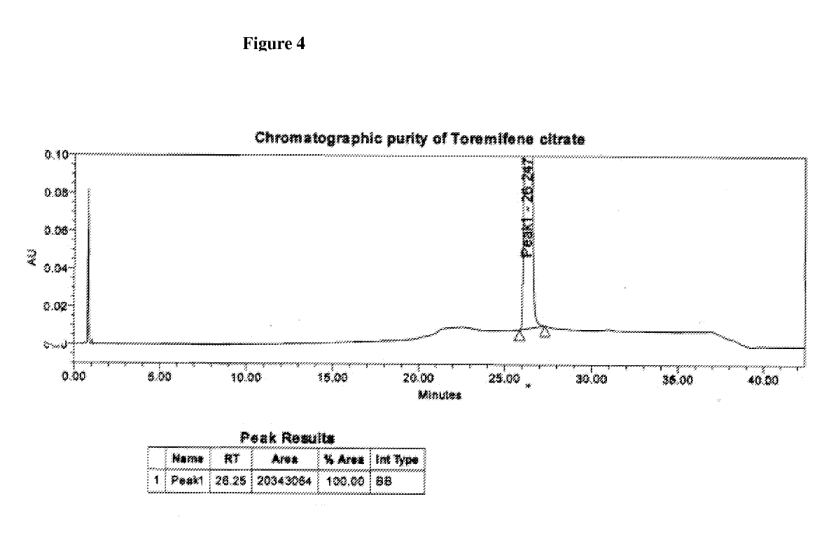

The polymorphic Form II of toremifene citrate as obtained is having HPLC purity greater than about 99.9% as shown in FIG. 4. The polymorphic Form II of toremifene citrate as obtained is in the form of Z isomer, which is free from E isomer.

The polymorphic Form II of toremifene citrate as obtained can be used for preparing a pharmaceutically acceptable composition with pharmaceutically acceptable carrier. The polymorphic Form II of the toremifene citrate as described in the invention may be used for the preparation of a pharmaceutical composition with a carrier, for example, in the form of pharmaceutical preparations for parenteral, oral and intravenous administration.

The polymorphic Form II of the toremifene citrate as described in the invention may be used for the treatment or prevention of hormone dependent tumors. The method includes administering to a mammal, a therapeutically effective amount of toremifene citrate substantially as polymorphic Form II.

Another aspect of the present invention provides an improved process for the preparation of the Z-isomer of toremifene base and its pharmaceutically acceptable salts. The process includes:
(a) converting 1,2-diphenyl-1[4-[2-N,N-dimethylamino)ethoxy]-phenyl-1-butane1,4-diol to toremifene base without isolation of any intermediates;
(b) purification of crude toremifene base in a ketonic solvent; and
(c) isolating the Z-isomer of toremifene base, which is optionally converted to pharmaceutically acceptable salts.

In another embodiment of the present invention there is provided a process for the preparation of Z-isomer of the toremifene base and its pharmaceutically acceptable salts. The process includes converting 1,2-diphenyl-1[4-[2-N,N-dimethylamino)ethoxy]-phenyl-1-butane1,4-diol to toremifene base without isolation of the 4-hydroxy-1,2-diphenyl-1[4-2-N,N-dimethylamino)ethoxy]-phenyl-1-butene intermediate.

According to another embodiment, the present invention provides a process for the preparation of the Z-isomer of the toremifene base and its pharmaceutically acceptable salts. The process includes converting 1,2-diphenyl-1[4-[2-N,N-dimethylamino)ethoxy]-phenyl-1-butane1,4-diol to toremifene base without isolation of the 4-acetoxy-1,2-diphenyl-1[4-2-N,N-dimethylamino)ethoxy]-phenyl-1-butene intermediate

The present invention also provides for a process for the preparation of Z-isomer of the toremifene base and its pharmaceutically acceptable salts. The process includes converting 1,2-diphenyl-1[4-[2-N,N-dimethylamino)ethoxy]-phenyl-1-butane1,4-diol to toremifene base without isolation of 4-acetoxy-1,2-diphenyl-1[4-[2-N,N-dimethylamino)ethoxy]-phenyl-1-butanol intermediate.

The preparation of toremifene base involves an insitu reaction of 1,2-diphenyl-1[4-[2-N,N-dimethylamino)ethoxy]-phenyl-1-butane1,4-diol with acetic anhydride in hydrocarbon solvents to give acetoxy derivative of the 1,2-diphenyl-1[4-[2-N,N-dimethylamino)ethoxy]-phenyl-1-butane1,4-diol at the primary hydroxyl group.

Examples of hydrocarbon solvents include hexane, heptane, n-heptane, octane, nonane, toluene, benzene, o, m or p-xylenes and/or mixtures thereof.

The reaction mixture may be stirred in the temperature range from about 70° C. to about 110° C. The stirring may be done for about 1 hour to about 3 hours. The stirring of the reaction mixture may be followed by the addition of acetyl chloride to the above stirred reaction mixture to give 4-acetoxy-1,2-diphenyl-1[4-[2-N,N-dimethylamino)ethoxy]-phenyl-1-butene. The addition of acetyl chloride to the above reaction mixture can be done in the temperature range from about 70° C. to about 110° C.

The reaction mixture containing 4-acetoxy derivative of 1,2-diphenyl-1[4-2-N,N-dimethylamino)ethoxy]-phenyl-1-butane-1,4-diol and acetyl chloride may be concentrated by any means known in the prior art, preferably it is concentrated under vacuum. The concentration of the reaction mixture under vacuum can be done in the temperature range from about 60° C. to about 85° C. The product obtained after concentration of the reaction mixture under vacuum may be further extracted with a water immiscible organic solvent and washed with an aqueous solution of an inorganic base, followed by vacuum condensation from about 60° C. to about 85° C.

Examples of water immiscible organic solvent may include esters, ethers, halogenated solvents, and aromatic hydrocarbons. Examples of aromatic hydrocarbon may include toluene, benzene, o, m or p-xylenes and/or mixtures thereof. Examples of esters may include ethylacetate, ethyl propionate, and ethyl butanoate. Examples of ethers may include diethylether, diisopropylether, ethylmethylether, tetrahydrofuran, and dioxane. Examples of halogenated solvents may include ethylene dichloride, dichloromethane, chloroform, and o, m & p-dichlorobenzenes. Examples of an inorganic base may include sodium hydroxide, potassium hydroxide, sodium carbonate, potassium carbonate, sodium bicarbonate, and potassium bicarbonate. Preferably, sodium carbonate is used.

The intermediate 4-acetoxy-1,2-diphenyl-1[4-[2-N,N-dimethylamino)ethoxy]-phenyl-1-butene obtained as oil may be further treated with an alkaline solution in an alcohol to provide 1,2-diphenyl-1[4-[2-N,N-dimethylamino)ethoxy]-phenyl-1-butene-4-ol as an oil, which can be used as such in the next step.

A suitable alcohol may include methanol, ethanol, isopropanol, isobutyl alcohol and/or their mixtures thereof. Examples of base may include sodium hydroxide, potassium hydroxide, sodium carbonate, potassium carbonate, sodium bicarbonate, and potassium bicarbonate. Preferably, sodium hydroxide is used.

The above reaction mixture may be heated from about 60° C. to the reflux temperature for about 2 to about 6 hours. The reaction mixture may be cooled to room temperature followed by an adjustment of pH with acid. The acid used for pH adjustment may include an inorganic acid such as hydrochloric acid, sulphuric acid, phosphoric acid, phosphorus acid, hydrobromic acid or hydrofluoric acid. Preferably, hydrochloric acid is used.

The above reaction mixture may be concentrated by any means known in the prior art including evaporation and distillation; preferably the reaction mixture is concentrated under vacuum.

The concentration of the reaction mixture under vacuum can be done in the temperature range from about 50° C. to about 60° C. The product obtained after concentration of the reaction mixture under vacuum may be further extracted with a water immiscible organic solvent.

Examples of water immiscible organic solvent may include esters, ethers, halogenated solvents, and aromatic hydrocarbons. Preferably, esters are used. Examples of aromatic hydrocarbon may include toluene, benzene, and o, m or p-xylenes. Examples of esters may include ethyl acetate, ethyl propionate, and ethyl butanoate. Examples of ethers may include diethylether, diisopropylether, ethylmethylether, tetrahydrofuran, and dioxane. Examples of halogenated solvents may include ethylene dichloride, dichloromethane, chloroform, and o, m & p-dichlorobenzenes.

The extracted mixture may be treated with activated carbon and filtered through hyflobed, which can be further concentrated under vacuum to give 1,2-diphenyl-1[4-2-N,N-dimethylamino)ethoxy]-phenyl-1-butene-4-ol as oil. The compound 1,2-diphenyl-1[4-[2-N,N-dimethylamino)ethoxy]-phenyl-1-butene-4-ol, which was obtained as oil, may be further treated with a chlorinating agent in a halogenated solvent. Examples of chlorinating agents may include thionyl chloride, phosphorous pentachloride or phosphorous oxytrichloride. Examples of halogenated solvents may include ethylene dichloride, chloroform, dichloromethane, carbon tetrachloride and o, m & p-dichlorobenzenes.

The reaction mixture may be heated from about 40° C. to about the reflux temperature of the solvent. The reaction mixture may be heated for about 5 to about 9 hours.

After heating, the reaction mixture may be concentrated by any means known in the prior art, such as, evaporation and distillation; preferably reaction mixture is concentrated under vacuum. The residue so obtained may be extracted with a water immiscible organic solvent. Examples of water immiscible organic solvent may include esters, ethers, halogenated solvents, and aromatic hydrocarbons. Examples of aromatic hydrocarbon may include toluene, benzene, and o, m or p-xylenes. Examples of esters may include ethylacetate, ethyl propionate, and ethyl butanoate. Examples of ethers may include diethylether, diisopropylether, ethylmethylether, tetrahydrofuran, and dioxane. Examples of halogenated solvents may include ethylene dichloride, dichloromethane, chloroform, and o, m & p-chlorobenzenes.

The aqueous solution of the base can be used to adjust the pH of the extracted mixture.

Examples of base may include sodium hydroxide, potassium hydroxide, sodium carbonate, potassium carbonate, sodium bicarbonate, potassium bicarbonate, and ammonia. Preferably, ammonia is used.

The oily residue obtained after concentration under vacuum and pH adjustment may be dissolved in a ketone for purification and the solid toremifene base can be isolated after cooling from about −10° C. to about 10° C.

Examples of ketones include aliphatic or alicyclic ketones. Examples of aliphatic ketones may include acetone, 2-pentanone, 3-pentanone, methylisobutyl ketone, and methyl ethyl ketone.

Examples of alicyclic ketones may include cyclopentanone, and cyclohexanone.

The purification process may be repeated to get the desired product, i.e., Z isomer of the toremifene base free of E isomer. The toremifene base so obtained may be dried using air drying, vacuum drying or any other drying methods known in the prior art. The drying temperature may be from about 40° C. to about 55° C. or any other range depending upon the drying technique. The toremifene base obtained by the above process is having purity greater than about 99.9% when analyzed by HPLC.

HPLC may utilize different types of stationary phase (for example, hydrophobic saturated carbon chains), a pump that moves the mobile phase(s) and analyte through the column (for example, Kromasil® C18 (150×4.6)mm, 5μ), and a detector that provides a characteristic retention time for the analyte.

The toremifene base obtained by the above process is the Z isomer which is free from E isomer. The term “Z isomer which is free from E isomer” refers to Z isomer wherein E isomer is absent.

The toremifene base obtained by the above process can be converted to its pharmaceutically acceptable salts, particularly polymorphic Form II of the toremifene citrate by dissolving it in a ketone followed by the addition of citric acid.

The aminoethoxy benzophenone precursor for the preparation of 1,2-diphenyl-1[4-[2-N,N-dimethylamino)ethoxy]-phenyl-1-butane1,4-diol can be prepared as illustrated in the example or by any other process known in the prior art.

In the following section embodiments are described by way of examples to illustrate the process of invention. However, these are not intended in any way to limit the scope of present invention. Several variants of these examples would be evident to persons skilled in the art.

### EXAMPLES

### Example 1

### Preparation of Toremifene Base

Step 1: Preparation of aminoethoxy benzophenone

A mixture of 4-hydroxy benzophenone (100 gm; 0.505 mole), 2-(dimethylamino)ethyl chloride hydrochloride (166 gm; 1.15 mole), potassium carbonate (400 gm) and acetone (1.5 liter) was refluxed at 56-57° C. for about 12 hours. Acetone was removed completely under vacuum, then de-ionized water (1.0 liter) and toluene (1.0 liter) were added to the above residue. The product was extracted in toluene and washed with 5% sodium hydroxide solution and de-ionized water. Toluene was removed under reduced pressure and the product was collected as oil and used as such in the next step.

Step 2: Preparation of 1,2-diphenyl-1[4-[2-N,N-dimethylamino)ethoxy]-phenyl-1-butane1,4-diol

Cinnamaldehyde (84.64 gm; 0.641 mole) in 230 mL of tetrahydrofuran was charged to a mixture of lithium aluminium hydride (15.26 gm; 0.401 mole) in 200 mL of tetrahydrofuran at 25-35° C. The reaction mixture was stirred at 30±2° C. for about 30 minutes. A solution of aminoethoxy benzophenone (entire batch from step 1) in 230 mL was charged into the reaction mixture at 30-40° C. followed by stiffing of the reaction mixture for about 2-3 hours. The reaction mixture was cooled to room temperature and poured into a 25% ammonium chloride solution (900 mL), filtered and the organic layer was separated. The aqueous layer was extracted with tetrahydrofuran (230 mL) and concentrated under vacuum. The residue was dissolved in toluene (1.1 liter) at 105-110° C., cooled to 25-30° C. and stirred for 3 hours at the same temperature; followed by isolation of the solid which was filtered, washed with toluene (300 mL) and dried in air oven at 65-70° C.

Yield: 1.45 wt/wt (71%)
Step 3: Preparation of 4-acetoxy-1,2-diphenyl-1[4-[2-N,N-dimethylamino)ethoxy]-phenyl-1-butanol

A mixture containing 1,2-diphenyl-1[4-[2-N,N-dimethylamino)ethoxy]-phenyl-1-butane1,4-diol (100 gm; 0.247 mole) and acetic anhydride (105 gm; 1.03 mole) in toluene was stirred at 90-95° C. for about 3 hours.

Step 4: Preparation of 4-acetoxy-1,2-diphenyl-1[4-[2-N,N-dimethylamino)ethoxy]-phenyl-1-butene

A solution of acetyl chloride (82.5 gm; 1.05 mole) in toluene (100 mL) was added to the 4-acetoxy-1,2-diphenyl-1[4-[2-N,N-dimethylamino)ethoxy]-phenyl-1-butanol (entire batch from step 3) in toluene at 90-100° C. in 30 minutes. The solution was stirred at the same temperature for 3 hours followed by concentration of the reaction mixture at 70-75° C. under vacuum. The product was extracted in toluene (700 mL), washed with 10% sodium carbonate solution (500 mL) and again concentrated at 70-75° C. under vacuum to give 4-acetoxy-1,2-diphenyl-1[4-[2-N,N-dimethylamino)ethoxy]-phenyl-1-butene as oil.

Step 5: Preparation of 4-hydroxy-1,2-diphenyl-1[4-[2-N,N-dimethylamino)ethoxy]-phenyl-1-butene

A mixture of 4-acetoxy-1,2-diphenyl-1[4-[2-N,N-dimethylamino)ethoxy]-phenyl-1-butene (entire batch obtained from step-4) in methanol (500 mL) and a 20% solution of sodium hydroxide (100 mL) was refluxed at 70-72° C. for 4 hours. The reaction mixture was cooled to room temperature and pH was adjusted to 8.0-8.5 with dilute hydrochloric acid, followed by concentration of the reaction mixture at 50-55° C. under vacuum. The product was extracted in ethylacetate (700 mL) at pH 8.1-8.3, charcolized with active carbon (10 gm), filtered through hyflobed, followed by further washing with ethylacetate (200 mL). The filtrate was concentrated to remove the solvent under vacuum at 60-65° C. giving 4-hydroxy-1,2-diphenyl-1[4-[2-N,N-dimethylamino)ethoxy]-phenyl-1-butene as oil.

Step-6: Preparation of toremifene Base

To a mixture of 4-hydroxy-1,2-diphenyl-1[4-[2-N,N-dimethylamino)ethoxy]-phenyl-1-butene (entire batch obtained from step-5) in dichloromethane (300 mL) thionyl chloride (58.76 gm; 0.494 mole) was added slowly at 20-30° C. followed by refluxing at 40-42° C. for about 7 hours. The reaction mixture was concentrated at 35-45° C. under vacuum and the residue was taken in a mixture of de-ionized water (600 mL) and ethylacetate (1.0 liter). The product was extracted in ethylacetate after adjusting the pH to 8.1-8.3 with 25% ammonia solution. The solvent was recovered under vacuum at 50-55° C. and the oily residue obtained after solvent recovery was dissolved in acetone (300 mL) and the product was isolated by filtration after cooling to 0-5° C. The wet solid was purified twice by dissolving in acetone followed by crystallization at 0-5° C. Toremifene base obtained after purification was dried at 40-45° C. in air oven. Yield: 40 gm HPLC purity: 100%

### Example 2

### Preparation of Crystalline Form of toremifene citrate

To a solution of toremifene base (40.0 gm) in acetone (480 mL) a solution of citric acid (24 gm) dissolved in acetone (120 mL) was added at 20-25° C. over 30-60 minutes. The mixture was further stirred at the same temperature for 2 hours to precipitate the solid, which was filtered and dried under vacuum at 45-50° C. to give toremifene citrate as a white crystalline solid. Yield: 50 gm HPLC purity: 100%

### Example 3

### Preparation of toremifene citrate (as per the '949 Patent)

To a solution of toremifene base (5.0 gm) in acetone (21.5 mL) a solution of citric acid (3.0 gm) was added in acetone (12.3 mL) at 40-45° C.; followed by stiffing of the reaction mixture to precipitate the solid. The slurry was then cooled to 20-25° C. and filtered. The wet solid was dried under vacuum to provide toremifene citrate.

Yield: 6.8 gm HPLC purity: 100%

XRPD was determined by using PANalytical X′ Pert Pro X-Ray Powder Diffractometer.

DSC was recorded on Perkin Elmer (Diamond, DSC).

IR was recorded on Perkin Elmer spectrum one FT-IR spectrometer.

We claim:

1. Polymorphic Form II of toremifene citrate.

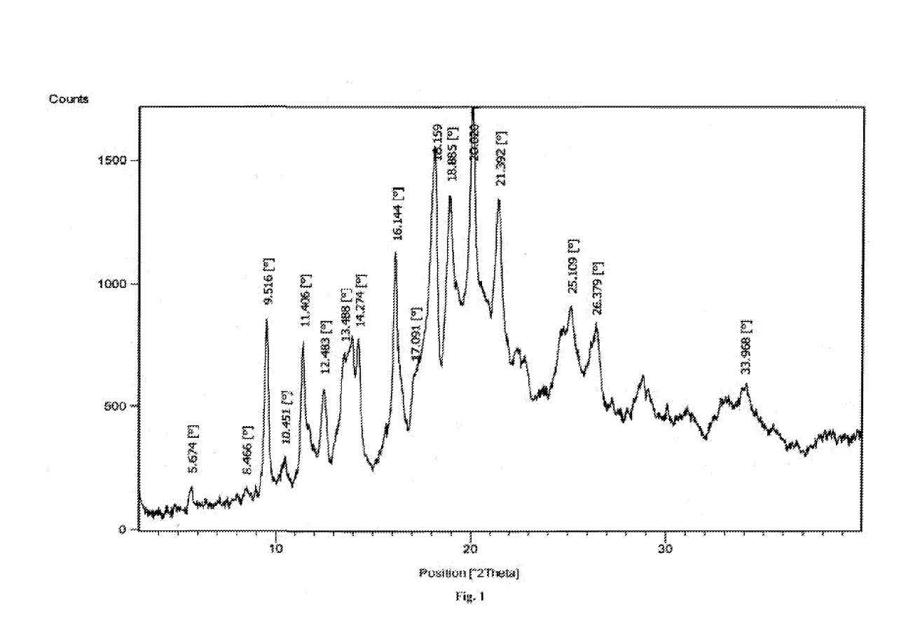

2. Polymorphic Form II of toremifene citrate comprising substantially the XRPD pattern as depicted in FIG. 1.

3. Polymorphic Form II of toremifene citrate comprising X-ray diffraction peaks expressed in degrees two-theta at 18.15, 18.88, 20.02, 21.39±0.2 in XRPD.

4. The polymorphic Form II of toremifene citrate of claim 3, further comprising X-ray diffraction peaks expressed in degrees two-theta at 5.67, 8.46, 9.51, 10.45, 11.40, 12.48, 13.48, 14.27, 16.14, 17.09, 25.10, 26.37, 33.96±0.2 in XRPD.

5. Polymorphic Form II of toremifene citrate having purity greater than 99.9% measured by HPLC area percentage as shown in FIG. 4.

6. Polymorphic Form II of toremifene citrate in Z isomer which is free of E isomer.

7. A pharmaceutically acceptable composition comprising polymorphic Form II of toremifene citrate and one or more pharmaceutically acceptable carriers.

8. A method of treating or preventing hormone dependent tumors comprising administering to a mammal a pharmaceutical composition comprising a therapeutically effective amount of polymorphic Form II of toremifene citrate.

9. A process for the preparation of polymorphic Form II of toremifene citrate, comprising:
(a) treating toremifene base with a ketone at an ambient temperature to obtain a solution;
(b) adding citric acid to the solution of step (a); and
(c) isolating polymorphic Form II of the toremifene citrate.

10. The process of claim 9, wherein the ketone comprises one or more of aliphatic or alicyclic ketones.

11. The process of claim 10 wherein the aliphatic ketones comprise acetone, 2-pentanone, 3-pentanone, methylisobutyl ketone or methyl ethyl ketone

12. The process of claim 10, wherein the alicyclic ketones comprise cyclopentanone or cyclohexanone.

13. A process for the preparation of the Z-isomer of toremifene base or its pharmaceutically acceptable salts comprising the steps of:
(a) converting 1,2-diphenyl-1[4-[2-N,N-dimethylamino)ethoxy]-phenyl-1-butane1,4-diol to toremifene base without isolation of any intermediates;
(b) purification of crude toremifene base in ketonic solvent; and
(c) isolating the Z-isomer of toremifene base, which is optionally converted to pharmaceutically acceptable salts.

14. The process of claim 13, wherein the ketone comprises aliphatic or alicyclic ketones.

15. The process of claim 14, wherein the aliphatic ketone comprises acetone, 2-pentanone, 3-pentanone, methylisobutyl ketone or methyl ethyl ketone

16. The process of claim 14, wherein the alicyclic ketone comprises cyclopentanone or cyclohexanone.

17. The process according to claim 13 wherein the 1,2-diphenyl-1[4-[2-N,N-dimethylamino)ethoxy]-phenyl-1-butane1,4-diol is converted to toremifene base without isolation of 4-hydroxy-1,2-diphenyl-1[4-[2-N,N-dimethylamino)ethoxy]-phenyl-1-butene intermediate.

18. The process according to claim 13 wherein the 1,2-diphenyl-1[4-[2-N,N-dimethylamino)ethoxy]-phenyl-1-butane1,4-diol is converted to toremifene base without isolation of 4-acetoxy-1,2-diphenyl-1[4-[2-N,N-dimethylamino)ethoxy]-phenyl-1-butene intermediate.

19. The process according to claim 13 wherein the 1,2-diphenyl-1[4-[2-N,N-dimethylamino)ethoxy]-phenyl-1-butane-1,4-diol is converted to toremifene base without isolation of 4-acetoxy-1,2-diphenyl-1[4-[2-N,N-dimethylamino)ethoxy]-phenyl-1-butanol intermediate.

20. Toremifene base of purity greater than 99.9% as measured by HPLC area percentage.

21. Z isomer of toremifene base which is free of E isomer.
